SEQUENCE LISTING (1 sequence)

SEQ ID NO: 1 - protein, 285 aa, Homo sapiens
KEEDVLKFLAAGTHLGGTNLDFQMEQYIYKRKSDGIYIINLKRTWEKLLVAARAIVAIENPADVSVISSRNTGQRAVLKFAAATGATPIAGRFTPGTFTNQIQAAFREPRLLVVTDPRADHQPLTEASYVNLPTIALCNTDSPLRYVDIAIPCNNKGAHSVGLMWWMLAREVLRMRGTISREHPWEVMPDLYFYRDPEEIEKEEQAAAEKAVTKEEFQGEWTAPAPEFTATQPEVADWSEGVQVPSVPIQQFPTEDWSAQPATEDWSAAPTAQATEWVGATTDWS

(19) United States
(12) Patent Application Publication
(10) Pub. No.: US 2007/0258897 A1
(43) Pub. Date: Nov. 8, 2007

(54) Antibodies Against Bacterial Antigens and Their Use in the Generation of Immune Responses Against Apoptotic Cells

(75) Inventors: Andrew Devitt, Edinburgh (GB); Christopher Gregory, Edinburgh (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: University Court of the University of Edinburgh, Edinburgh (GB)

(21) Appl. No.: 11/630,444

(22) PCT Filed: Jun. 23, 2005
(86) PCT No.: PCT/GB05/02489

§ 371 Date: Jun. 20, 2007

(30) Foreign Application Priority Data
Jun. 23, 2004 (GB) 0414055.4

(51) Int. Cl. A61K 39/395; A61K 48/00; A61P 43/00; G01N 33/53; G01N 33/567; G01N 33/574

(52) U.S. Cl. 424/9.1; 424/155.1; 435/7.1; 435/7.2; 435/7.21; 435/7.23

(57) ABSTRACT

The present invention provides a method of enhancing an immune response to apoptotic cells, the method comprising administration of a specific binding member which specifically binds to a microbial antigen. Also provided are pharmaceutical compositions comprising such antibodies and assays to identify specific binding members capable of enhancing an immune response to apoptotic cells. Also described are methods for the treatment of cancer using antibodies which bind to an apoptotic cell intracellular antigen.

The present invention relates to specific binding members and their use in therapy. In particular, the invention relates to specific binding members which bind to microbial ligands and the use of such specific binding members in the treatment of disease, for example, neoplastic disease.

### BACKGROUND TO THE INVENTION

An important function of macrophages is the engulfment of cells undergoing apoptosis via mechanisms that are normally both anti-inflammatory and immunosuppressive [Henson, 2001; Savill, 2002]. In certain categories of non-Hodgkin's lymphomas (NHL), most noted being Burkitt's lymphoma (BL), and including to a variable degree other NHL classes (eg immunoblastic lymphoma, pre-B and pre-T lymphoblastic lymphoma and thymoma [Berard, 1969; Harris, 1995; van Meyel, 1998; Hori, 2001; Zenger, 2002]), tumour-cell apoptosis occurs at relatively high rates and tumour-associated macrophages (TAMs)—or ‘starry sky’ macrophages as they are often described in this context—appear to engulf the dying tumour cells efficiently [Berard, 1969; Harris, 1995; Hori, 2001]. It is believed that, in such cases, apoptotic cells can potently dampen anti-tumour immune responses. Published evidence lends support to this notion: apoptotic lymphoma cells are poorly immunogenic and macrophages pulsed with apoptotic lymphoma cells fail to activate anti-tumour T-cell immunity in vivo [Ronchetti, 1999].

### SUMMARY OF THE INVENTION

The present inventors have surprisingly shown that antibodies raised against microbial ligands can bind apoptotic cells and can switch macrophage response to apoptotic cells from an anti-inflammatory/immunosuppressive response to a pro-inflammatory/immunostimulatory response. Moreover, the inventors have further shown that, in vivo, such antibodies can significantly slow or halt tumour growth.

Thus, according to a first aspect of the present invention, there is provided a method of enhancing an immune response to apoptotic cells, said method comprising administration of a specific binding member which specifically binds to a microbial antigen.

The demonstration that anti-microbial antibodies can enhance the immune response to apoptotic cells facilitates, for the first time, the use of such antibodies in the treatment of cancer.

Accordingly, in a second aspect of the present invention, there is provided a method of treating cancer comprising administration of a therapeutically effective amount of a specific binding member or a nucleic acid encoding said specific binding member which specifically binds to a microbial antigen to a subject in need thereof.

In the context of the present application, the subject may be any animal, preferably a mammal, most preferably a human.

In a third aspect, there is provided the use of (i) a specific binding member which specifically binds to a microbial antigen or (ii) a nucleic acid encoding said specific binding member in the preparation of a medicament for the treatment of cancer.

In a fourth aspect, there is provided (i) a specific binding member or (ii) a nucleic acid encoding said specific binding member which specifically binds to a microbial antigen for use in the treatment of cancer.

The present invention also provides in a fifth aspect a pharmaceutical composition for the treatment of cancer, wherein the composition comprises a specific binding member which specifically binds to a microbial antigen or a nucleic acid encoding said specific binding member.

The demonstration that antibodies raised against microbial ligands can bind apoptotic cells also enables for the first time the use of such antibodies in the identification of apoptotic cells. The use of such antibodies as diagnostic agents in the identification of apoptotic cells is thus encompassed by the invention.

Accordingly, in a sixth aspect of the invention, there is provided a method of diagnosing the presence of apoptotic cells in a biological sample, said method comprising the steps:

(a) bringing into contact a specific binding member which specifically binds to a microbial antigen and a biological sample;

(b) determining the presence of binding of the specific binding member to cells of the biological sample; wherein the presence of binding of the specific binding member to cells of the biological sample is indicative that the cells being bound are apoptotic.

Such an assay may be utilised, for example, in the monitoring of response of tissues to therapy. Some cancers are associated with high levels of apoptotic cells. Thus in a seventh aspect of the invention, there is provided a method for monitoring the progression of a cancer in a patient, comprising the steps of:

(a) contacting a biological sample from a patient at a first point in time with specific binding member which specifically binds to a microbial antigen;

(b) detecting in the sample an amount of specific binding member that binds to the binding agent;

(c) repeating steps (a) and (b) using a biological sample obtained from the patient at a subsequent point in time; and

(d) comparing the amount of binding member detected in step (c) to the amount detected in step (b) and therefrom monitoring the progression of the cancer in the patient.

Any suitable biological samples may be used in the method of the sixth or seventh aspect of the invention. Such samples may be from any tissue and may be, for example, biopsy samples.

Certain diagnostic assays may be performed in vivo directly on a tumour. Thus, in an eighth aspect of the invention, there is provided a method of detecting the presence of apoptotic cells in tissue in vivo, said method comprising the steps(a) bringing into contact a specific binding member which specifically binds to a microbial antigen and a said tissue in vivo;

(b) determining the presence of binding of the specific binding member to cells of the tissue; wherein the presence of binding of the specific binding member to cells of the tissue is indicative that the cells being bound are apoptotic.

The assay may be used to determine the presence of cancer cells. A change in the amount of apoptotic cells in a tumour tissue may be indicative of growth of the tumour. In some other cancers, it may be indicative of regression of the tumour.

The present invention may also be used to monitor the response to therapy in vivo. Thus the invention further provides in a ninth aspect a method for monitoring the progression of a cancer in a patient, comprising the steps of:

(a) contacting a tissue in vivo at a first point in time with a specific binding member which specifically binds to a microbial antigen;

(b) detecting in the amount of specific binding member that binds to the tissue;

(c) repeating steps (a) and (b) at a subsequent point in time; and

(d) comparing the amount of binding member detected in step (c) to the amount detected in step (b) and therefrom monitoring the progression of the cancer in the patient.

Binding of the specific binding member may be detected directly or indirectly using any method known in the art. For example, the antibody may be detected via a reporter group. The methods may involve linking the antibody to radioisotopes, paramagnetic labels, echogenic liposomes, or other appropriate agents that can be detected by imaging methods, and injected into the host intravenously. After an appropriate time, imaging can be performed, either whole body for diagnostic purposes or locally at specific sites, such as carotid artery, in a quantitative manner to assess the hosts response to a treatment regimen.

Such binding agents may also be used in histological applications. Alternatively, polynucleotide probes may be used within such applications.

Suitable specific binding members which may be used in the methods of the invention include any specific binding member which has binding specificity for a microbial ligand and which has cross-reactivity with an apoptotic cell epitope.

In one embodiment of the invention, the specific binding member is selected from mAb wn1 222-5 (Cambridge Bioscience Ltd, Cambridge, UK), mAb 15174 (QED Bioscience, Inc., San Diego, Calif. 92127), mAb 15306 (QED Bioscience, Inc., San Diego, Calif. 92127), mAb 15308 (QED Bioscience, Inc., San Diego, Calif. 92127), mAb 983 (Chemicon Europe, Ltd., Chandlers Ford, Hampshire, United Kingdom), mAb 995 (Chemicon Europe, Ltd., Chandlers Ford, Hampshire, United Kingdom), mAb 756 (Chemicon Europe, Ltd., Chandlers Ford, Hampshire, United Kingdom), and mAb 746 MAB 8598 (Chemicon Europe, Ltd., Chandlers Ford, Hampshire, United Kingdom).

In preferred embodiments of the invention, the specific binding member has binding specificity for a ligand which binds CD14. Suitable CD14 ligands may include LPS, lipotecheic acid, peptidoglycan and lipoarabinomannan. In a particularly preferred embodiment, the ligand which binds CD14 is the bacterial lipopolysaccharide endotoxin, LPS.

For example, a preferred specific binding member for use in the present invention is mAb wn1 222-5 (Cambridge Bioscience Ltd, Cambridge, UK), which has binding specificity for LPS.

Another preferred specific binding member for use in the present invention is mAb 15174 (QED Bioscience, Inc., San Diego, Calif. 92127), which has binding specificity for LPS.

Another preferred specific binding member for use in the present invention is mAb 15306 (QED Bioscience, Inc., San Diego, Calif. 92127), which has binding specificity for LPS.

A particularly preferred specific binding member for use in the present invention is mAb 15308 (QED Bioscience, Inc., San Diego, Calif. 92127), which has binding specificity for LPS. As described in the Examples below, the inventors have evidence that this antibody may be cross-reactive with laminin binding protein (LBP) in apoptotic cells. Moreover, the antibody has been shown to induce pro-inflammatory responses in macrophages. Accordingly, in further preferred embodiments of the invention, the specific binding member has binding specificity for laminin binding protein (LBP).

The demonstration that such antibodies bind to apoptotic cells and induce an immunostimulatory response is further surprising in that LBP is considered to be an intracellular molecule. As the general consensus in the art dictates that apoptotic cells are cleared by macrophages before the cells become leaky, it is surprising that antibodies which target intracellular antigens can switch macrophages from an anti-inflammatory to a pro-inflammatory mode. Without being bound by any one theory, it is believed that LBP may be externalised in apoptotic cells. Additionally or alternatively, it is possible that the cells become leaky before being cleared and that the antibodies are binding the LBP target intracellularly.

The discovery that such intracellular antigens in apoptotic cells may be bound by antibodies suggests that other intracellular antigens may be used to target apoptotic cells.

Accordingly, in a tenth independent aspect of the invention, there is provided a method of enhancing an immune response to apoptotic cells, said method comprising administration of a specific binding member which specifically binds to an intracellular antigen.

As with the anti-microbial antibodies described above, the antibodies with binding specificity for intracellular antigens may be used in methods of treating cancer.

Accordingly, in an eleventh aspect of the invention, there is provided a method of treating cancer, said method comprising administration of a therapeutically effective amount of a specific binding member which specifically binds to an apoptotic cell intracellular antigen to a subject in need thereof.

In a twelfth aspect, there is provided the use of (i) a specific binding member which specifically binds to an apoptotic cell intracellular antigen or (ii) a nucleic acid encoding said specific binding member in the preparation of a medicament for the treatment of cancer.

In a thirteenth aspect, there is provided a specific binding member which specifically binds to an apoptotic cell intracellular antigen for use in the treatment of cancer.

The present invention also provides, as a fourteenth aspect of the invention, a pharmaceutical composition for the treatment of cancer, wherein the composition comprises a specific binding member which specifically binds to an apoptotic cell intracellular antigen.

Suitable specific binding members for use in the, tenth, eleventh, twelfth, thirteenth and fourteenth aspects of the invention need not necessarily be cross-reactive with microbial antigens. Suitable specific binding members for use in these aspects of the invention may include specific binding members having binding specificity for intracellular antigens such as actin, phosphatidyl serine, other cytoskeletal components or annexin I.

The invention further provides assays for identification of further agents, for example antibodies, that can be used in the enhancement of an immune response to apoptotic cells and which can optionally be used in the treatment of cancer.

Accordingly, in an fifteenth aspect of the invention, there is provided an assay method for identification of an antibody capable of enhancing an immune response to apoptotic cells, said assay method comprising the steps:
a) bringing into contact a specific binding member with binding specificity for a microbial antigen with an apoptotic cell; and
b) determining the absence or presence of cross-reactivity of the specific binding member with the apoptotic cell, wherein the presence of cross-reactivity of the specific binding member with the apoptotic cell is indicative of ability to enhance an immune response against said apoptotic cells.

In preferred embodiments of the sixteenth aspect of the invention, the assay method of the invention may include the further step (c) bringing together (i) apoptotic cells and the specific binding member determined in step (b) to show crossreactivity with the apoptotic cells and (ii) innate immune cells, and determining the induction of an immunostimulatory response, for example a pro-inflammatory response, and/or inhibition of an immunosuppressive response, for example an anti-inflammatory response, in the innate immune cells.

Any suitable innate immune cells may be used. For example, the innate immune cells may be macrophages, neutrophils or dendritic cells. In one embodiment, the innate immune cells are macrophages.

In a seventeenth aspect of the invention, there is provided of an assay method for identification of a specific binding member capable of enhancing an immune response to apoptotic cells, said assay method comprising steps:
(a) providing a specific binding member with binding specificity for a microbial antigen
(b) bringing into contact said specific binding member with binding specificity for a microbial antigen, apoptotic cells and macrophages, and
(c) determining the induction of an immunostimulatory response, for example a pro-inflammatory response, and/or inhibition of an immunosuppressive response, for example an anti-inflammatory response, in the macrophages, wherein the presence of an immunostimulatory response and/or inhibition of an immunosuppressive response is indicative of ability to enhance an immune response against said apoptotic cells.

The assays of the sixteenth and seventeenth aspects of the invention may further comprise step(d) identifying the apoptotic cell epitope to which the antibody binds.

### DETAILED DESCRIPTION

Specific Binding Members

As used herein, a “binding member” is a member of a pair of structures which have binding specificity for one another. The binding member may be naturally derived or wholly or partially synthetically produced. In particular, the present invention is concerned with antigen-antibody type reactions, although a binding member of the invention and for use in the invention may be any moiety which can bind to a microbial antigen and/or an intracellular antigen. Thus specific binding members for use in the present invention may include, for example, bacteriophage receptors which can interact with bacterial antigens, for example an LPS antigen, and which can cross react with apoptotic cell antigens, e.g. CD14 ligands.

Antibodies

In the context of the present invention, an “antibody” should be understood to refer to an immunoglobulin or part thereof or any polypeptide comprising a binding domain which is, or is homologous to, an antibody binding domain. Antibodies include but are not limited to polyclonal, monoclonal, monospecific, polyspecific antibodies and fragments thereof and chimeric antibodies comprising an immunoglobulin binding domain fused to another polypeptide.

Intact antibodies comprise an immunoglobulin molecule consisting of heavy chains and light chains, each of which carries a variable region designated VH and VL, respectively. The variable region consists of three complementarity determining regions (CDRs, also known as hypervariable regions) and four framework regions (FR) or scaffolds. The CDR forms a complementary steric structure with the antigen molecule and determines the specificity of the antibody.

Fragments of antibodies may retain the binding ability of the intact antibody and may be used in place of the intact antibody. Accordingly, for the purposes of the present invention, unless the context demands otherwise, the term “antibodies” should be understood to encompass antibody fragments. Examples of antibody fragments include Fab, Fab′, F (ab′)2, Fd, dAb, and Fv fragments, scFvs, bispecific scFvs, diabodies, linear antibodies (see U.S. Pat. No. 5,641,870, Example 2; Zapata et al., Protein Eng 8 (10): 1057-1062 [1995]); single-chain antibody molecules; and multispecific antibodies formed from antibody fragments.

The Fab fragment consists of an entire L chain (VL and CL), together with VH and CH1. Fab′ fragments differ from Fab fragments by having additional few residues at the carboxy terminus of the CH1 domain including one or more cysteines from the antibody hinge region. The F (ab′) 2 fragment comprises two disulfide linked Fab fragments.

Fd fragments consist of the VH and CH1 domains.

Fv fragments consist of the VL and VH domains of a single antibody.

Single-chain Fv fragments are antibody fragments that comprise the VH and VL domains connected by a linker which enables the scFv to form an antigen binding site. (see Pluckthun in The Pharmacology of Monoclonal Antibodies, vol.113, Rosenburg and Moore eds., Springer-verlag, New York, pp. 269-315 (1994).

Diabodies are small antibody fragments prepared by constructing scFv fragments (see preceding paragraph) with short linkers (about 5-10 residues) between the VH and VL domains such that inter-chain but not intra-chain pairing of the V domains is achieved, resulting in a multivalent fragment, i.e. a fragment having two antigen-binding sites (see, for example, EP 404 097; WO 93/11161; and Hollinger et al., Proc.Natl. Acad. Sci. USA, 90:6444-6448 (1993))

Further encompassed by fragments are individual CDRs.

As described above, the present invention is not limited to use of the specific sequences of the antibody, the VH, VL and the CDRs thereof as described herein but also extends to variants thereof which (i) maintain specificity for a microbial antigen and show crossreactivity with an apoptotic cell epitope and/or (ii) maintains binding specificity for an apoptotic cell intracellular antigen. Thus, the CDR amino acid sequences in which one or more amino acid residues are modified may also be used as the CDR sequence. The modified amino acid residues in the amino acid sequences of the CDR variant are preferably 30% or less, more preferably 20% or less, most preferably 10% or less, within the entire CDR. Such variants may be provided using the teaching of the present application and techniques known in the art. The CDRs may be carried in a framework structure comprising an antibody heavy or light chain sequence or part thereof. Preferably such CDRs are positioned in a location corresponding to the position of the CDR(s) of naturally occurring VH and VL domains. The positions of such CDRs may be determined as described in Kabat et al, Sequences of Proteins of Immunological Interest, US Dept of Health and Human Services, Public Health Service, Nat'l Inst. of Health, NIH Publication No. 91-3242, 1991 and online at http://immuno.bme.nwu.edu.

Furthermore, modifications may alternatively or additionally be made to the Framework Regions of the variable regions. Such changes in the framework regions may improve stability and reduce immunogenicity of the antibody.

Thus, such variants which may be used in the invention include fragments of an antibody or of a polypeptide which comprise a stretch of amino acid residues of at least 5 to 7 contiguous amino acids, often at least about 7 to 9 contiguous amino acids, typically at least about 9 to 13 contiguous amino acids, more preferably at least about 20 to 30 or more contiguous amino acids and most preferably at least about 30 to 40 or more consecutive amino acids.

Variants or use in the invention may include an antibody or polypeptide modified by varying the amino acid sequence of the protein, e.g. by manipulation of the nucleic acid encoding the protein or by altering the protein itself. Such derivatives of the natural amino acid sequence may involve insertion, addition, deletion and/or substitution of one or more amino acids, preferably while providing a peptide having apoptotic cell opsonisation activity. Preferably such derivatives involve the insertion, addition, deletion and/or substitution of 25 or fewer amino acids, more preferably of 15 or fewer, even more preferably of 10 or fewer, more preferably still of 4 or fewer and most preferably of 1 or 2 amino acids only.

The monoclonal antibodies which may be used include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81: 6851-6855 (1984)). Chimeric antibodies of interest herein include “primatized” antibodies comprising variable domain antigen-binding sequences derived from a non-human primate(e. g. Old World Monkey, Ape etc), and human constant region sequences.

The term “antibody” includes antibodies which have been “humanised”. Methods for making humanised antibodies are known in the art. Methods are described, for example, in Winter, U.S. Pat. No. 5,225,539. A humanised antibody may be a modified antibody having the hypervariable region of a monoclonal antibody and the constant region of a human antibody. Thus the binding member may comprise a human constant region.

The variable region other than the hypervariable region may also be derived from the variable region of a human antibody and/or may also be derived from a monoclonal antibody such as mAb15308. In such case, the entire variable region may be derived from monoclonal antibody mAb15308 and the antibody is said to be chimerised. Methods for making chimerised antibodies are known in the art. Such methods include, for example, those described in U.S. patents by Boss (Celltech) and by Cabilly (Genentech). See U.S. Pat. Nos. 4,816,397 and 4,816,567, respectively.

In preferred embodiments of the invention, the binding member binds to laminin binding protein.

The percent identity of two amino acid sequences or of two nucleic acid sequences may be determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilised for the comparison of sequences is the algorithm of Myers & Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis & Robotti (1994) Comput. Appl. Biosci., 10 :3-5; and FASTA described in Pearson & Lipman (1988) Proc. Natl. Acad. Sci. 85:2444-8. Within FASTA, ktup is a control option that sets the sensitivity and speed of the search.

Where high degrees of sequence identity are present there will be relatively few differences in amino acid sequence. Thus for example they may be less than 20, less than 10, or even less than 5 differences.

Production of Binding Members

Specific binding members of and for use in the present invention may be produced in any suitable way, either naturally or synthetically. Such methods may include, for example, traditional hybridoma techniques (Kohler and Milstein (1975) Nature, 256:495-499), recombinant DNA techniques (see e.g. U.S. Pat. No. 4,816,567), or phage display techniques using antibody libraries (see e.g. Clackson et al. (1991) Nature, 352: 624-628 and Marks et al. (1992) Bio/Technology, 10: 779-783). Other antibody production techniques are described in Antibodies: A Laboratory Manual, eds. Harlow et al., Cold Spring Harbor Laboratory, 1988.

Traditional hybridoma techniques typically involve the immunisation of a mouse or other animal with an antigen in order to elicit production of lymphocytes capable of binding the antigen. The lymphocytes are isolated and fused with a a myeloma cell line to form a hybridoma cells which are then cultured in conditions which inhibit the growth of the parental myeloma cells but allow growth of the antibody producing cells. The hybridoma may be subject to genetic mutation, which may or may not alter the binding specificity of antibodies produced. Synthetic antibodies can be made using techniques known in the art (see, for example, Knappik et al, J. Mol. Biol. (2000) 296, 57-86 and Krebs et al, J. Immmunol. Meth. (2001) 2154 67-84.

Modifications may be made in the VH, VL or CDRs of the binding members, or indeed in the FRs using any suitable technique known in the art. For example, variable VH and/or VL domains may be produced by introducing a CDR, e.g. CDR3 into a VH or VL domain lacking such a CDR. Marks et al. (1992) Bio/Technology, 10: 779-783 describe a shuffling technique in which a repertoire of VH variable domains lacking CDR3 is generated and is then combined with a CDR3 of a particular antibody to produce novel VH regions. Using analogous techniques, novel VH and VL domains comprising CDR derived sequences may be produced.

Alternative techniques of producing variant antibodies of the invention may involve random mutagenesis of gene(s) encoding the VH or VL domain using, for example, error prone PCR (see Gram et al, 1992, P.N.A.S. 89 3576-3580. Additionally or alternatively, CDRs may be targeted for mutagenesis e.g. using the molecular evolution approaches described by Barbas et al 1991 PNAS 3809-3813 and Scier 1996 J Mol Biol 263 551-567.

Having produced such variants, antibodies and fragments may be tested for binding to microbial antigens, apoptotic cells or intracellular antigens.

The antibodies of and for use in the invention may comprise further modifications. For example the antibodies can be glycosylated, pegylated, or linked to albumin or a nonproteinaceous polymer.

Antibodies of and for use in the invention may be labelled. Labels which may be used include radiolabels, enzyme labels such as horseradish peroxidase or alkaline phosphatase, or biotin.

Nucleic Acid

Nucleic acid of and for use in the present invention may comprise DNA or RNA. It may be produced recombinantly, synthetically, or by any means available to those in the art, including cloning using standard techniques.

The nucleic acid may be inserted into any appropriate vector. A vector comprising a nucleic acid of the invention forms a further aspect of the present invention. In one embodiment the vector is an expression vector and the nucleic acid is operably linked to a control sequence which is capable of providing expression of the nucelic acid in a host cell. A variety of vectors may be used. For example, suitable vectors may include viruses (e. g. vaccinia virus, adenovirus,etc.), baculovirus); yeast vectors, phage, chromosomes, artificial chromosomes, plasmids, or cosmid DNA.

The vectors may be used to introduce the nucleic acids of the invention into a host cell. A wide variety of host cells may be used for expression of the nucleic acid of the invention. Suitable host cells for use in the invention may be prokaryotic or eukaryotic. They include bacteria, e.g. E. coli, yeat, insect cells and mammalian cells. Mammalian cell lines which may be used include Chinese hamster ovary cells, baby hamster kidney cells, NSO mouse melanoma cells, monkey and human cell lines and derivatives therof and many others.

A host cell strain that modulates the expression of, modifies, and/or specifically processes the gene product may be used. Such processing may involve glycosylation, ubiquiination, disulfide bond formation and general post-translational modification.

The present inventors have shown that antibodies directed to certain microbial antigens, for example microbial CD14 ligands, for example 15308, are cross-reactive with apoptotic cell antigens, and such antibodies and fragments and derivatives thereof can be used as cancer therapeutics to make tumour cells susceptible to immmune system attack.

The binding members may be administered alone or in combination with one or more further agents. Thus, the present invention further provides products comprising a specific binding member, which binds to a microbial antigen and is crossreactive with an apoptotic cell antigen, and an active agent as a combined preparation for simultaneous, separate or sequential use in the treatment of cancer. Active agents may include chemotherapeutic agents including, Doxorubicin, taxol, 5-Fluorouracil (5 FU), Leucovorin, Irinotecan, Mitomycin C, Oxaliplatin, Raltitrexed, Tamoxifen and Cisplatin which may operate synergistically with the binding member of the present invention. Other active agents may include suitable doses of pain relief drugs such as non-steroidal anti-inflammatory drugs (e.g. aspirin, paracetamol, ibuprofen or ketoprofen) or opiates such as morphine, or anti-emetics. In further embodiments, the active agent may be a further binding member. Thus, in preferred embodiments the binding member may be administered in combination with one or more further binding members. Such binding members may include but are not limited to an anti-CD20 antibody e.g Rituxan (Rituximab)(Biogen IDEC (Cambridge, Mass., USA); an anti-VEGF antibody e.g. Avastin(bevacizumab), Genentech (South San Francisco, Calif., USA)/Roche (Basel, Switzerland); an anti-CD171A antibody, e.g. Panorex(edrecolomab) Centocor (Malvern, Pa., USA)/Glaxo SmithKline (Uxbridge, UK); an anti-CEA anti-idiotypic mAb e.g. CeaVac, Titan Pharmaceuticals (South San Francisco, Calif., USA); an anti-EGFR antibody e.g. Erbitux(cetuximab), ImClone(New York, USA)/Bristol Myers Squibb (New York, USA), Merck (Whitehouse Station, N.J., USA); an anti-HMFG anti-idiotypic mAb e.g TriAb, Titan Pharmaceuticals (South San Francisco, Calif., USA), an anti-EGFR antibody e.g. ABX-EGF, Abgenix (Fremont, Calif., USA) /Amgen Thousand Oaks, Calif.) and/or an anti-HER2 antibody e.g. Herceptin, Genentech (South San Francisco, Calif., USA).

Preferably, the active agent synergises with the binding member to enhance tumour killing. The binding member of the invention may carry a detectable label.

Treatment

“Treatment” includes any regime that can benefit a human or non-human animal. The treatment may be in respect of an existing condition or may be prophylactic (preventative treatment). Treatment may include curative, alleviation or prophylactic effects.

“Treatment of cancer” includes treatment of conditions caused by cancerous growth and includes the treatment of neoplastic growths or tumours. Examples of tumours that may be treated by the system of the invention are, for instance, sarcomas, including osteogenic and soft tissue sarcomas, carcinomas, e.g., breast-, lung-, bladder-, thyroid-, prostate-, colon-, rectum-, pancreas-, stomach-, liver-, uterine-, cervical and ovarian carcinoma, lymphomas, including Hodgkin and non-Hodgkin lymphomas, neuroblastoma, melanoma, myeloma, Wilms tumor, and leukemias, including acute lymphoblastic leukaemia and acute myeloblastic leukaemia, gliomas- and retinoblastomas.

The compositions and methods of the invention may be particularly useful in the treatment of existing cancer and in the prevention of the recurrence of cancer after initial treatment or surgery.

Administration

Binding members of and for use in the present invention may be administered alone but will preferably be administered as a pharmaceutical composition, which will generally comprise a suitable pharmaceutical excipient, diluent or carrier selected dependent on the intended route of administration.

Binding members of and for use in the present invention may be administered to a patient in need of treatment via any suitable route. The precise dose will depend upon a number of factors, including the precise nature of the member (e.g. whole antibody, fragment or diabody), and the nature of any detectable label attached to the member.

Some suitable routes of administration include (but are not limited to) oral, rectal, nasal, topical (including buccal and sublingual), vaginal or parenteral (including subcutaneous, intramuscular, intravenous, intradermal, intrathecal and epidural) administration. Intravenous administration is preferred.

It is envisaged that injections (intravenous) will be the primary route for therapeutic administration of the compositions although delivery through a catheter or other surgical tubing may also be envisaged. Liquid formulations may be utilised after reconstitution from powder formulations.

For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The composition may also be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919; EP-A-0058481) copolymers of L-glutamic acid and gamma ethyl-L-glutamate (Sidman et al, Biopolymers 22(1): 547-556, 1985), poly (2-hydroxyethyl-methacrylate) or ethylene vinyl acetate (Langer et al, J. Biomed. Mater. Res. 15: 167-277, 1981, and Langer, Chem. Tech. 12:98-105, 1982). Liposomes containing the polypeptides are prepared by well-known methods: DE 3,218, 121A; Epstein et al, PNAS USA, 82: 3688-3692, 1985; Hwang et al, PNAS USA, 77: 4030-4034, 1980; EP-A-0052522; E-A-0036676; EP-A-0088046; EP-A-0143949; EP-A-0142541; JP-A-83-11808; U.S. Pat. Nos. 4,485,045 and 4,544,545. Ordinarily, the liposomes are of the small (about 200-800 Angstroms) unilamellar type in which the lipid content is greater than about 30 mol. % cholesterol, the selected proportion being adjusted for the optimal rate of the polypeptide leakage.

Examples of the techniques and protocols mentioned above and other techniques and protocols which may be used in accordance with the invention can be found in Remington's Pharmaceutical Sciences, 16^{th} edition, Oslo, A. (ed), 1980.

The composition may be administered in a localised manner to a tumour site or other desired site or may be delivered in a manner in which it targets tumour or other cells. Targeting therapies may be used to deliver the active agent more specifically to certain types of cell, by the use of targeting systems such as antibody or cell specific ligands. Targeting may be desirable for a variety of reasons, for example if the agent is otherwise unacceptably toxic, or if it would otherwise require too high a dosage, or if it would not otherwise be able to enter the target cells.

Dose

The compositions are preferably administered to an individual in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

The optimal dose can be determined by physicians based on a number of parameters including, for example, age, sex, weight, severity of the condition being treated, the active ingredient being administered and the route of administration. In general, a serum concentration of polypeptides and antibodies that permits saturation of receptors is desirable. A concentration in excess of approximately 0.1 nM is normally sufficient. For example, a dose of 100 mg/m² of antibody provides a serum concentration of approximately 20 nM for approximately eight days.

As a rough guideline, doses of antibodies may be given weekly in amounts of 10-300 mg/m². Equivalent doses of antibody fragments should be used at more frequent intervals in order to maintain a serum level at or in excess of the optimal concentration.

Assays

The invention further provides assays for identification of further agents, for example antibodies, that can be used in the enhancement of an immune response to apoptotic cells and which can optionally be used in the treatment of cancer.

Accordingly, in a further aspect of the invention, there is provided an assay method for identification of a specific binding member, for example antibody, capable of enhancing an immune response to apoptotic cells, said assay method comprising steps:
a) bringing into contact an specific binding member with binding specificity for a microbial antigen with an apoptotic cell; and
c) determining the absence or presence of cross-reactivity of the specific binding member with the apoptotic cell, wherein the presence of cross-reactivity of the specific binding member with the apoptotic cell is indicative of ability to enhance an immune response against said apoptotic cells.

In preferred embodiments, the assay method of the invention may include the further step (c) bringing together (i) apoptotic cells and antibody determined in step (b) to have crossreactivity with the apoptotic cells and (ii) macrophages, and determining an immunostimulatory response, for example a pro-inflammatory response, and/or inhibition of an immunosuppressive response, in the macrophages.

In a further aspect of the invention, there is provided an assay method for identification of an specific binding member capable of enhancing an immune response to apoptotic cells, said assay method comprising steps:
(a) providing an specific binding member with binding specificity for a microbial antigen
(b) bringing into contact said specific binding member with binding specificity for a microbial antigen, apoptotic cells and macrophages, and
(c) determining the induction of an immunostimulatory response, for example a pro-inflammatory response, and/or inhibition of an immunosuppressive response, for example an anti-inflammatory response, in the macrophages, wherein the presence of an immunostimulatory response and/or inhibition of an immunosuppressive response is indicative of ability to enhance an immune response against said apoptotic cells.

The assays of these aspects of the invention may further comprise step (d) selecting a candidate agent that displays cross-reactivity and/or induces an immunostimulatory response and/or inhibits an immunosuppressive response, and, optionally, step (e) identifying the apoptotic cell epitope to which the specific binding member binds.

The present invention further provides a screening method comprising the step of screening a library of antibodies each with binding specificity for a microbial antigen for the ability to bind an apoptotic cell epitope.

The assay of the invention may be a screen, whereby a number of candidate agents are tested. Accordingly, any suitable technique for screening compounds known to the person skilled in the art may be used. The screen may be a high-throughput screen. For example, WO84/03564 describes a method in which large numbers of peptides are synthesised on a solid substrate and reacted with an agent and washed. Bound entities are detected.

For example, a panel of antibodies (e.g. obtained from commercial sources) raised against a variety of microbial molecules, for example microbial molecules that are known to function as CD14-ligands, including LPS, lipoteichoic acid, peptidoglycan and lipoarabinomannan [Gregory, 1999; Dziarski, 2000] may be screened against viable and apoptotic human and murine lymphoma cell lines, including those in the animal models described below.

Antibody cross-reactivity may be assessed using any suitable method known in the art. Cross-reactivity may be assessed at various stages of apoptosis—prior to and following loss of plasma-membrane integrity—and particular attention can be paid to reactivity towards cell-surface versus intracellular components. In the latter event, reactivity with apoptotic cells can be compared with that of permeabilised viable cells. It should be noted that macrophage pattern recognition receptors (PRRs) may well interact with internal structures of apoptotic cells in addition to surface components since, in vitro, macrophages preferentially recognise late apoptotic cells, coincident with loss of plasma-membrane integrity [Devitt, 2003].

Judicious staining of normal and malignant primary cells and cell lines (e.g. primary human malignant lymphoma and leukaemia cells) will permit assessment of any preferential binding of antibodies to apoptotic tumour cells.

Apoptotic-cell reactive antibodies from these initial screens may be selected for further study.

Antibodies showing strong apoptotic-cell reactivity can be used as probes to identify molecules bearing the cross-reactive epitopes displayed by apoptotic cells.

For example, antibody-reactive proteins will be isolated by immunoprecipitation/immunoblotting and subjected to mass spectrometric analysis. Confirmation of antibody reactivity may be sought using recombinant forms of the candidate proteins—over-expressed in cells and produced as soluble molecules. If available, additional antibodies raised against the identified host molecules may also be tested for reactivity with apoptotic cells and may be included in the further investigations below as appropriate.

Multiple approaches may be taken to determine the relevance of the identified host molecules to apoptotic-cell clearance mechanisms: available antibodies to these structures, for example prepared as F(ab′)₂ fragments, may be tested for their ability to inhibit interaction of apoptotic cells with macrophages using standard in vitro assays. Soluble recombinant forms of these proteins may be similarly tested.

Since it is hypothesised that many of the cross-reactive host proteins are CD14-ligands, it can be determined whether antibodies binding these proteins inhibit the interaction of soluble CD14 with apoptotic cells as described [Devitt, 2004].

Using membrane-anchored CD14 on established stable transfectant cell lines, or soluble CD14 in an ELISA-based approach, can be ascertained the extent to which the candidate proteins, in soluble form, can interact directly with CD14 can be ascertained.

In vitro Macrophage Responses

Specific binding members, for example antibodies, selected from the studies outlined above can be tested initially for their ability to opsonise apoptotic lymphoma cells such that they switch macrophage reactivity to these apoptotic cells to a more immunostimulatory response, e.g. from an anti-inflammatory to a pro-inflammatory response. For example, opsonisation of apoptotic human BL and murine λ-MYC lymphoma cells [Kovalchuk, 2000] with murine IgG antibodies from the selected panel may be tested.

The ability of specific binding members to induce an immunostimulatory response (which should be understood to include induction of a new response or enhancement of an exiting response) and the ability of specific binding members to inhibit an immunosuppressive response (which should be understood to include complete inhibition or partial inhibition of an existing response) can be tested using any method known in the art. For example, the specific binding members can be tested for their capacity to activate inflammatory responses in macrophages assessed by measurement of a variety of molecules including arachidonic acid metabolites, H₂O₂ and pro-inflammatory cytokines such as TNF-α. Inhibition of an immunosuppressive response may be measured, for example, by measurement of a reduction in anti-inflammatory cytokine production e.g. of one or more of TGF-β; IL-10 etc.

Whether such opsonised cells activate macrophage tumouricidal activity in vitro can also be determined. Comparisons can be made between (1) opsonising antibodies that have proven capacity to identify apoptotic cell associated molecular patterns (ACAMPs) and also block interactions between apoptotic cells and macrophages/CD14, and (2) those antibodies that identify ACAMPs but have no such antagonistic activity. Comparison may also be made between antibodies derived from the present studies and two additional available murine IgG mabs, one specifying phosphatidylserine (Upstate) the other Annexin I (Pharmingen) on apoptotic cells. Finally the effects on macrophage responsiveness of apoptotic cells opsonised with IgG antibody cocktails derived from the selected mAb panel may also be determined.

Therapeutic Activity of Selected Antibodies in vivo

Specific binding members, for example antibodies, displaying potent ability to activate macrophage immunostimulatory and/or tumouricidal effects and/or ability to inhibit immunosuppressive effects may be further tested using in vivo studies, for example in studies of anti-lymphoma therapy in mice. For example, models which can be used include transplanted human BL or murine λ-MYC lymphoma in SCID mice as well as spontaneous or transplanted murine λ-MYC lymphoma in immunocompetent animals. These models allow assessment of the relative roles of the innate and adaptive immune systems in mediating antibody therapy. Animals are given injections of candidate therapeutic antibodies (including cocktails thereof, as appropriate) following tumour development and tumour growth retardation and remission is sought. Histological analyses of regressing tumours can be used to seek lymphocyte infiltration of tumours and changes in TAM phenotype. In vitro measurements of anti-tumour CTL responses and of cytotoxic activity of TAMs can also be undertaken as a prelude to detailed mechanistic immunological studies (including, for example activation of dendritic cells). Optimal antibody dosages, mixtures and administration regimens may be determined empirically.

The invention also contemplates the use of competitive drug screening assays in which antibodies specific for microbial antigens and cross-reactive with an apoptotic cell epitope specifically compete with test agents for binding to said epitope.

Agents, antibodies etc identified by the screening method of the present invention and their use in the manufacture of a medicament for the treatment of cancer are also contemplated by the invention.

Preferred features of each aspect of the invention are as for each of the other aspects mutatis mutandis.

The invention will now be described further in the following non-limiting examples. Reference is made to the accompanying drawings in which:

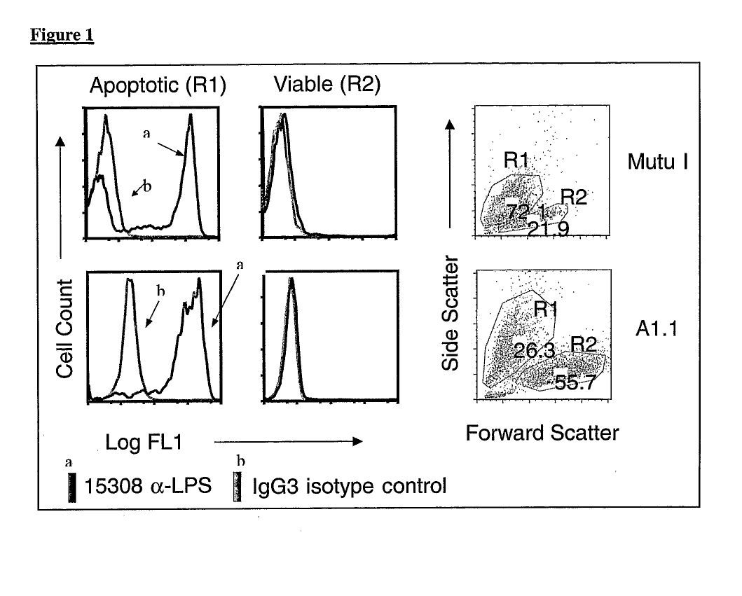

FIG. 1 shows cross-reactivity of apoptotic human and mouse lymphoma cells with anti-LPS mAb 15308.

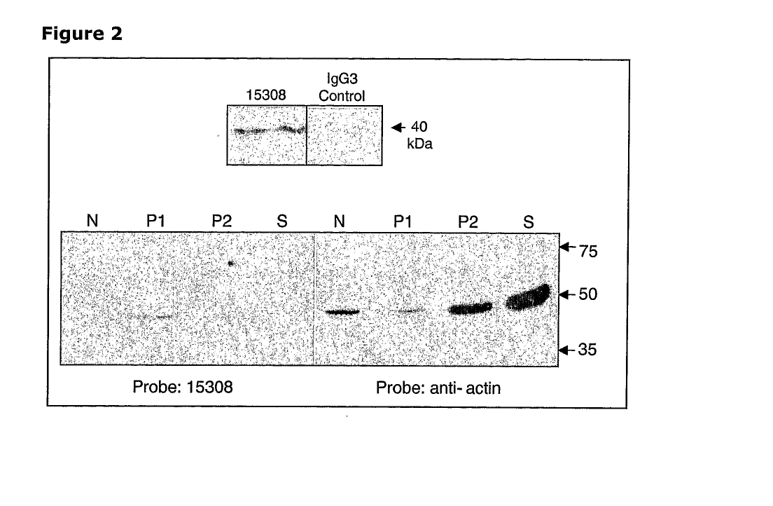

FIG. 2 illustrates the reactivity of human BL cell lysates with anti-LPS mAb 15308

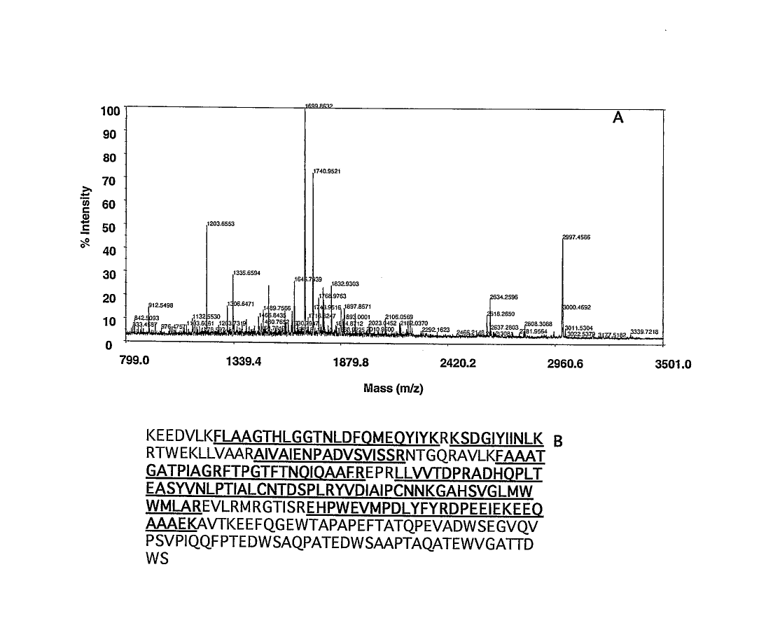

FIG. 3 illustrates matrix-assisted laser desorption and ionisation time-of-flight (MALDI-TOF) mass spectrometry of bands generated in FIG. 2 with mAb 15308.

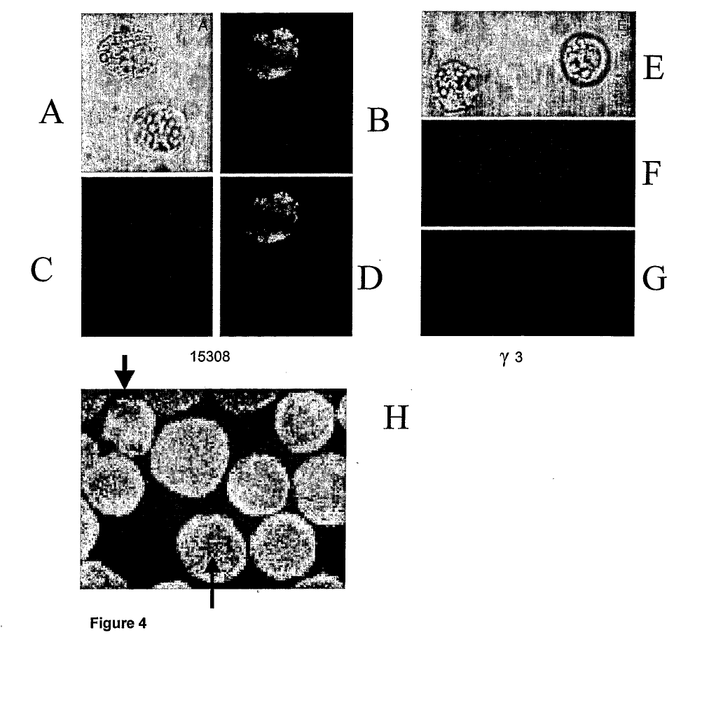

FIG. 4 illustrates binding of the 15308 antibody to a human B-cell lymphoma cell line, Mutu 1, as determined by confocal microscopy.

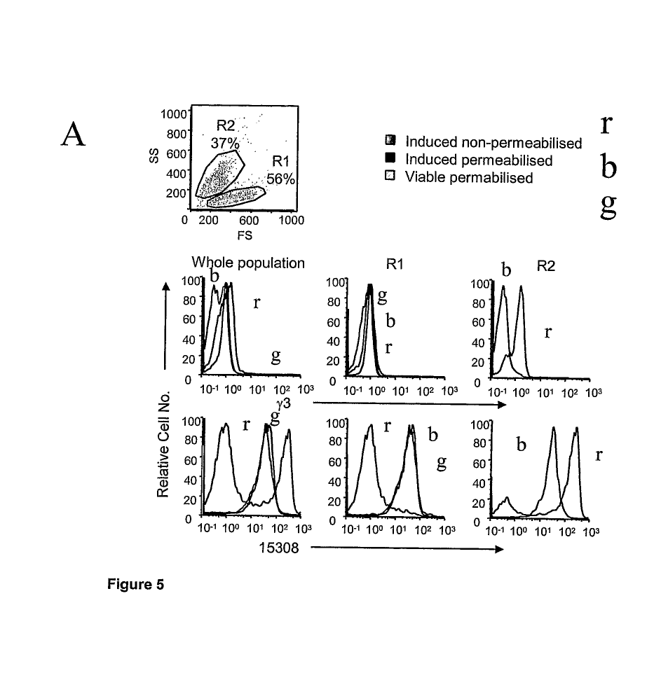

FIG. 5 illustrates analysis by flow cytometry of the ability of antibody 15308 to bind cells either fixed and permeabilised (using intrastain™, DAKO) or left intact.

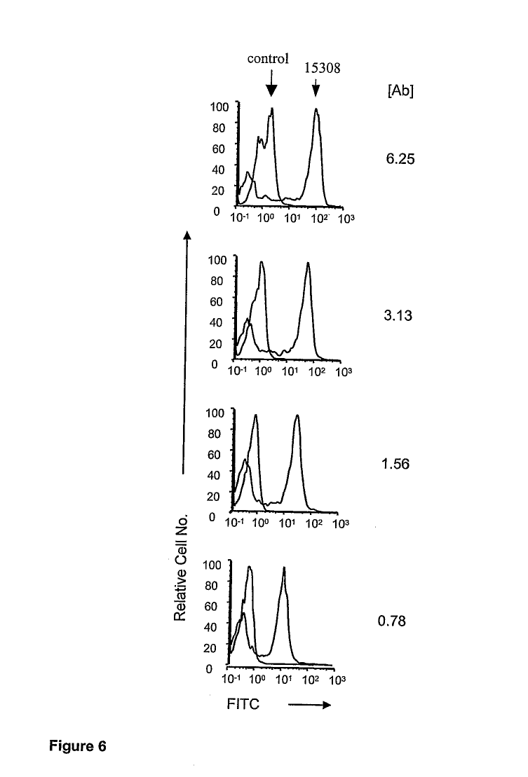

FIG. 6 illustrates the results of measurement of the sensitivity of anti-LPS antibody 15308 in binding to its epitope on apoptotic lymphoma cells.

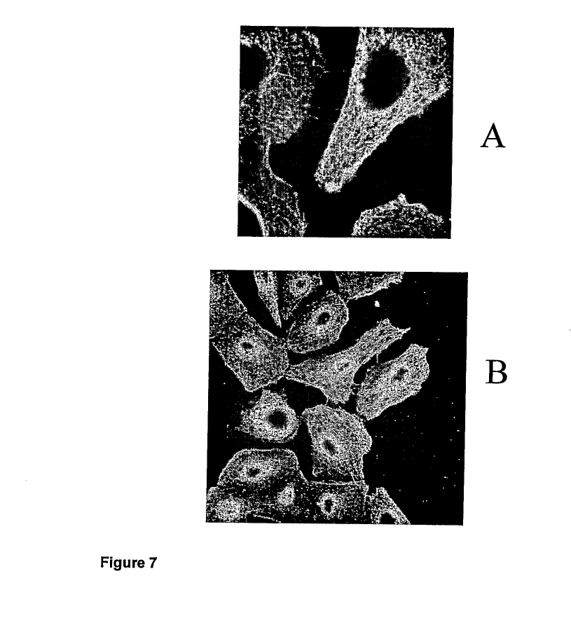

FIG. 7 illustrates binding of anti-LPS antibody, 15308 in adherent cell lines.

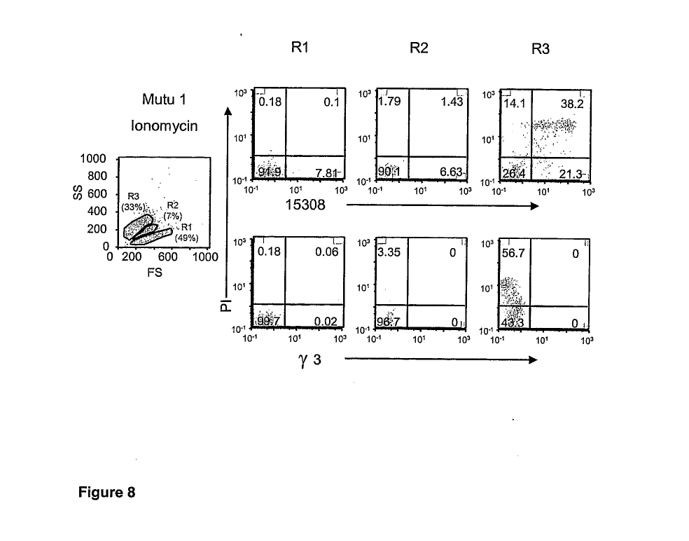

FIG. 8 illustrates flow cytometric analyses of Mutu I cells at various stages of apoptosis labelled with antibody mAb 15308.

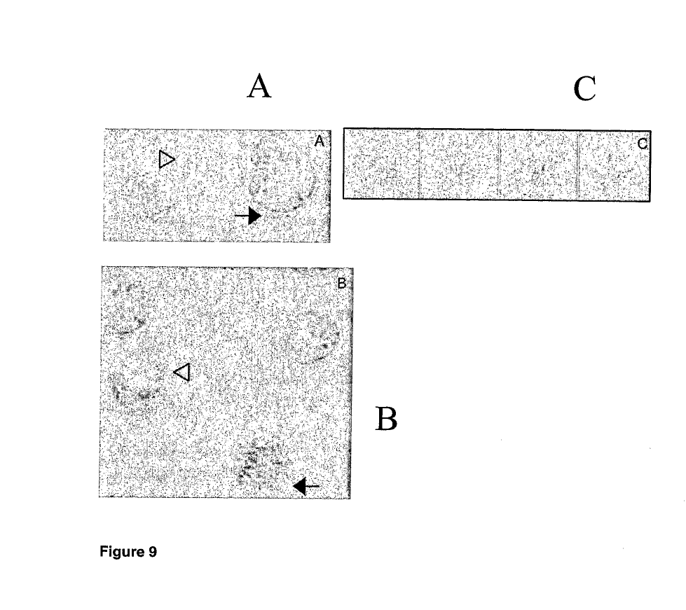

FIG. 9 shows confocal microscope photographs of apoptotic human lymphoma cells, showing that the 15308 epitope is exposed on the surface of the cells.

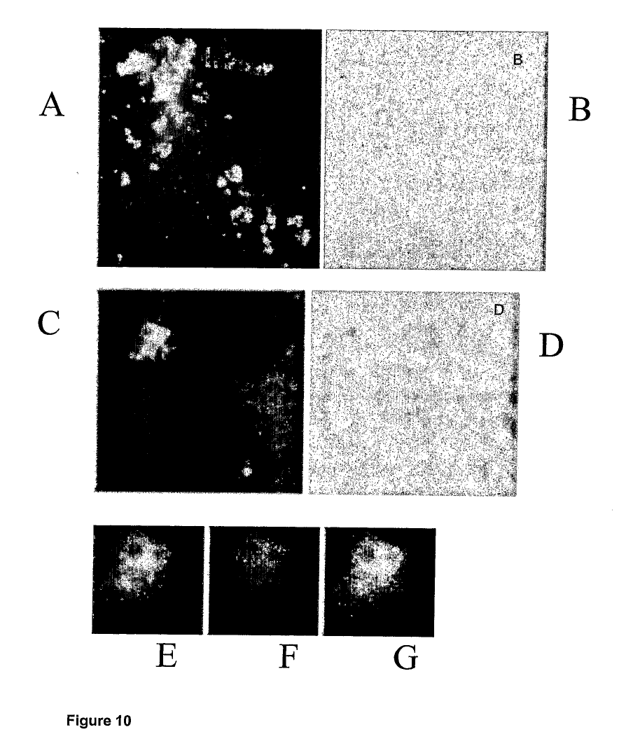

FIG. 10 shows confocal fluorescence images of the 15308 epitope visualised by goat anti-mouse secondary antibody labelled with alexaFluor-568 (red), biotinylated-PS visualised by streptavidin labelled with alexaFluor-488 (green).

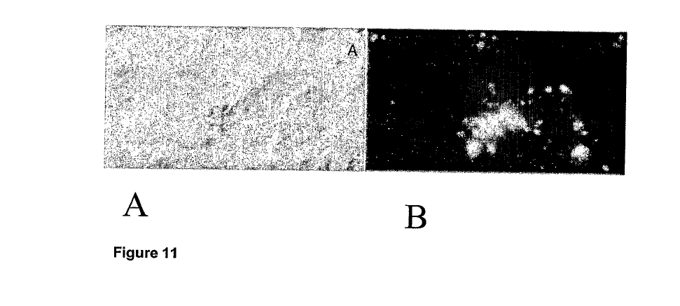

FIG. 11 shows under bright field (A) or a fluorescence image (B) of the 15308 epitope.

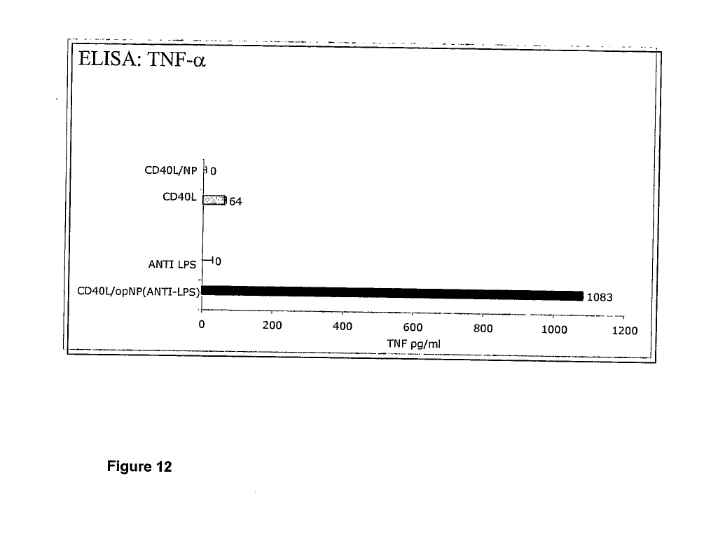

FIG. 12 shows the results of ELISA assay to monitor TNF-α production in macrophages in response to apoptotic cells in the absence and presence of mAb15308.

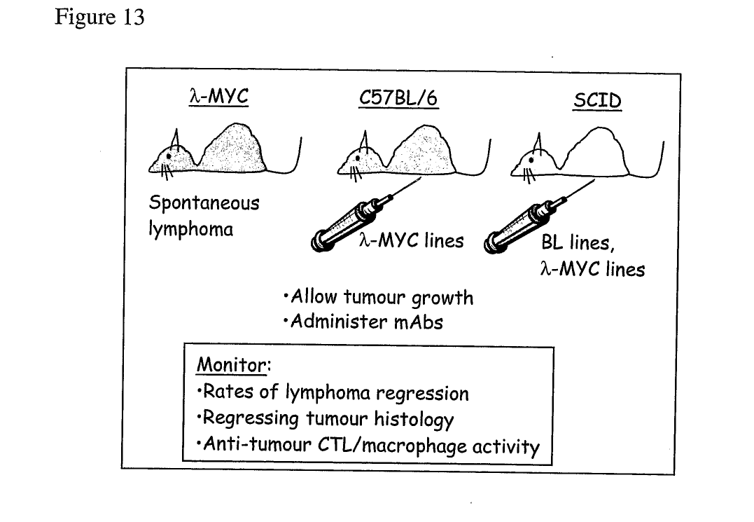

FIG. 13 is a schematic showing in vivo animal models.

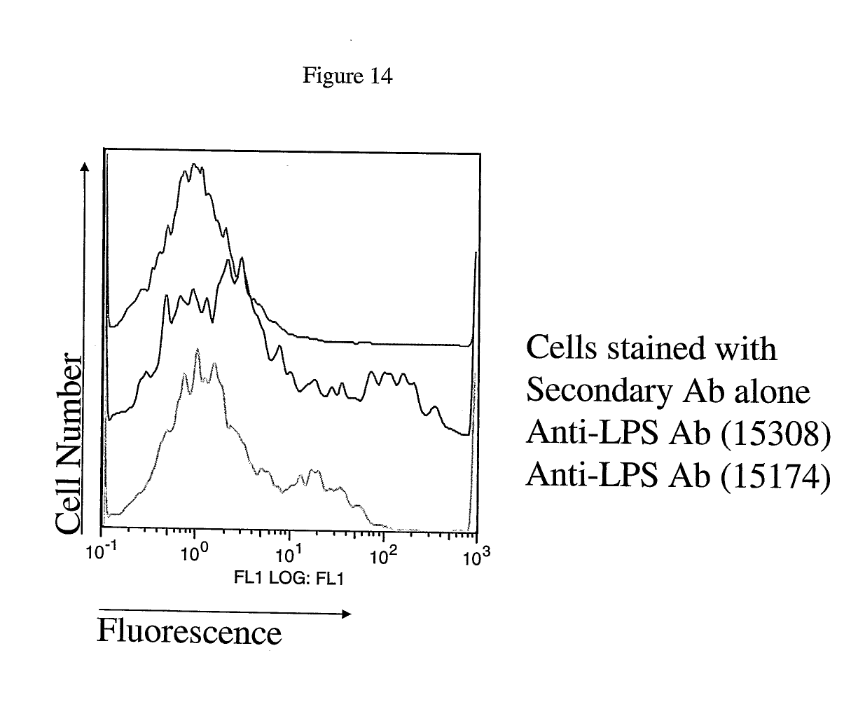

FIG. 14 shows flow cytometry results obtained from mouse primary thymic cells stained with anti-microbial antibodies.

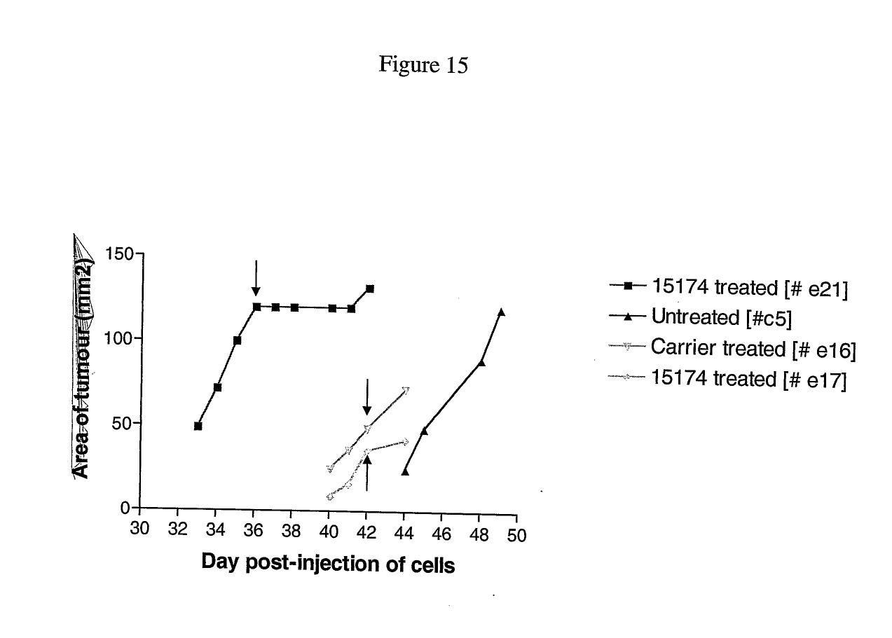

FIG. 15 shows the effects of intra-tumoural injection of mAb 15174 or PBS on tumour growth in vivo.

### EXAMPLE 1

A panel of 5 commercially available anti-LPS antibodies were tested for cross-reactivity with apoptotic cells. Apoptotic Mutu I BL cells were stained with the indicated panel of anti-LPS mAbs and analysed as shown in FIG. 1 for mAb 15308.

Briefly, flow cytometric analyses of human BL line Mutu I and murine thymoma line A1.1 were labelled with antibody (for example, in the example shown in FIG. 1, anti-LPS mAb 15308 (IgG3)) by indirect immunofluorescence. Mutu I cells were induced to undergo apoptosis with ionomycin; A1.1 cells underwent spontaneous apoptosis. Right-hand panels indicate light-scatter dot-plots enumerating cells falling into R1 (apoptotic) and R2 (viable) zones as described [Dive, 1992]. Left and central panels show fluorescence histograms of cells in apoptotic and viable zones, respectively.

The results for the tested commercially available antibodies are summarised in Tables 1A and 1B. In Table 1A, four of the antibodies tested were shown to react with apoptotic cells, including human and murine lymphoma cell lines. Of the panel of 8 antibodies shown in Table 1B, five of the antibodies tested were shown to react with apoptotic cells including human and murine lymphoma cell lines.

TABLE 1A
Summary of cross-reactivity of murine anti-
LPS mAbs with apoptotic human lymphoma cells

| | | | Cross-Reactivity | |
|---|---|---|---|---|
| | Microbial | | with Apoptotic | |
| mAb | Specificity | Isotype | Cells | Supplier |
| wn1 222-5 | LPS | IgG 2a | + | Cambridge |
| | | | | Bioscience |
| 15308 | LPS (various | IgG3 | + | QED |
| | serotypes) | | | Bioscience |
| 15174 | LPS (various | IgG2a | + | QED |
| | serotypes) | | | Bioscience |
| 15306 | LPS (various | IgG2a | + | QED |
| | serotypes) | | | Bioscience |
| 512F | Chlamydial | IgG2b | − | Dako |
| | LPS | | | |

TABLE 1B
Summary of cross-reactivity of murine anti-
LPS mAbs with apoptotic human lymphoma cells

| | | | | Cross |
|---|---|---|---|---|
| | | | | reactivity |
| mAb/clone | | | class/ | with |
| name | | | isotype | Apoptotic |
| (Supplier) | immunogen | specificity | (format) | Cells |
| HM2045/ | not | reacts with | IgM | negative |
| Clone 20 | stated | a large | (culture | |
| (HBT | | panel of | medium) | |
| (Hycult) | | LPS, alpha- | | |
| | | KDO | | |
| | | specific. | | |
| | | Does not | | |
| | | react with | | |
| | | beta-KDO or | | |
| | | 5-deoxy-KDO. | | |
| | | Does not | | |
| | | require | | |
| | | lipid-A to | | |
| | | bind. | | |
| HM2048/ | not | reacts with | IgG3 | negative |
| Clone 55 | stated | liptoteichoic | (culture | |
| (HBT | | acid. | medium) | |
| (Hycult) | | | | |
| MAB 983 | insoluble | 3D polmer of | IgG3 | positive |
| (Chemicon) | Peptidogly- | PG. Epitope = | (ascites) | |
| | can obtained | discontinous glycan | | |
| | by | +/or aa residues, | | |
| | TCA/heat | not fully | | |
| | and | defined. | | |
| | ethanol | Ineffective | | |
| | extraction | inhibitors = | | |
| | of | muramyldipeptide, | | |
| | Strptococcus | N-acetylglucos | | |
| | mutans | amine, | | |
| | BHT cells | chitin and | | |
| | | acid- | | |
| | | hydrolysed | | |
| | | chitin. | | |
| MAB 995 | insoluble | 3D polmer of | IgG1 | positive |
| (Chemicon) | Peptidogly- | PG. Epitope = | (ascites) | |
| | can obtained | discontinous glycan | | |
| | by | +/or aa residues, | | |
| | TCA/heat | not fully | | |
| | and | defined. | | |
| | ethanol | Ineffective | | |
| | extraction | inhibitors = | | |
| | of | muramyldipeptide, | | |
| | Strptococcus | N-acetylglucos | | |
| | mutans | amine, | | |
| | BHT cells | chitin and | | |
| | | acid- | | |
| | | hydrolysed | | |
| | | chitin. | | |
| mAB 756 | Steptococcus | strains EC1, | IgG1 | positive |
| (Chemicon) | | EC2, STR1, | (purified) | |
| | | STR2, W, | | |
| | | SGW, STAPH | | |
| | | EPI | | |
| MAB 746 | Salmonella | most boiled | IgG2a | weak |
| (Chemicon) | common | salmonella | (purified) | positive |
| | Ag | | | |
| MAB 8594 | V1680E | RSV-fusion | IgG 1k | negative |
| (Chemicon) | | protein | (ascites) | |
| MAB 8598 | A2 | RSV-fusion | IgG1k | positive |
| (Chemicon) | | protein | (ascites) | |

mAb 15308 was further tested. FIG. 2 shows the results of testing the reactivity of human BL cell lysates with anti-LPS mAb 15308. Mutu I BL cells (a mixture of apoptotic and viable) were lysed, electrophoresed on 10% polyacrylamide SDS gels and blotted on PVDF membranes with the indicated mAbs. In the upper panel, total cell lysates are shown. In the lower panels, lysates were fractionated by sequential centrifugation into 1,000 g pellet (N), 27,000 g pellet (P1), 100,000 g pellet (P2) and remaining supernatant (S).

Bands generated as in FIG. 2 with mAb 15308 were excised from the polyacrylamide gel and tryptic digests analysed by MALDI-TOF mass spectrometry (FIG. 3). Resultant peptide masses were subjected to SwissProt and MASCOT database searches. Lower panel summarises output from the database query, positively identifying the protein reactive with mAb 15308 as laminin-binding protein. The matched peptides covered 58% of the total protein as shown in red underlined.

Thus, the biochemical analyses (FIGS. 2, 3) indicate that mAb 15308, may specify laminin-binding protein, a multifunctional protein that is known to be involved in ribosomal biogenesis and, via plasma-membrane expression, cell adhesion to extracellular matrix [Kazmin, 2003]. Laminin-binding protein therefore represents a candidate ACAMP bearing cross-reactivity with the prototypic PAMP, LPS.

### EXAMPLE 2

### The 15308 Antibody Binds to an Intracellular-Epitope Found within both Viable and Apoptotic Cells

As shown in FIG. 4, the 15308 antibody binds to an intracellular-epitope found within both viable and apoptotic cells, as determined by confocal microscopy. The human B-cell lymphoma cell line, Mutu 1 was treated with ionomycin for 16h to undergo apoptosis (A-G). Binding of the anti-LPS antibody, 15308 was detected with goat anti-mouse secondary antibody labelled with alexaFluor-488 (green, D and B).

Cells were counterstained with the impermeant dyeTO-PRO-3 for visualisation of nucleic acid and as a measure the integrity of the membrane (blue, B and C).

Viable cells were not stained at all (lower cell, A). In contrast, the isotype-matched control antibody (γ3) exhibited no staining of the apoptotic cells (E,F and G).

When fixed and permeabilised (intrastain™, DAKO) Mutu 1 cells also exhibit cytoplasmic staining with 15308 antibody (H). Viable cells display large rounded nuclei(open arrow head) whereas the nuclei of apoptotic cells are condensed (closed arrow head).

### EXAMPLE 3

### Levels of 15308 Epitope Expression in both Permeabilised Apoptotic and Viable Lymphoma Cells

The human B-cell lymphoma cell line, Mutu 1 was treated with ionomycin for 16h (Induced) or left untreated (Viable).

Cells were then either fixed and permeabilised (using intrastain™, DAKO) or left intact. The ability of antibody 15308 to bind cells was analysed by flow cytometry. Staining with a gamma 3 isotype mouse monoclonal antibody (γ3) was performed as a negative control. The results are shown in FIG. 5. Within the population, cells were defined as either viable or apoptotic on the basis of light scatter properties; apoptotic (R1), viable (R2).

A slight decrease in fluorescence was observed for both 15308 and isotype antibody as a result of the permeabilisation process (blue (b) vs red (r) histogram, R2). Equivalent levels of 15308 epitope expression in both permeabilised apoptotic and viable lymphoma cells.

### EXAMPLE 4

### Determination of the Sensitivity of Anti-LPS Antibody 15308 in Binding to its Epitope on Apoptotic Lymphoma Cells

The human B-cell lymphoma cell line, Mutu 1 was treated with ionomycin for 16h. The ability of antibody 15308 to bind cells was analysed by flow cytometry. Staining with a gamma 3 isotype mouse monoclonal antibody (γ3) was performed at the same concentrations as a negative control. Bound antibody was detected with FITC labeled goat anti-mouse secondary antibody. The results are shown in FIG. 6.

Values represent the concentration at which primary antibodies ([Ab]) were incubated with cells (μg/ml). Cells were at a density of 2.5×10⁶/ml during incubation with primary antibody.

### EXAMPLE 5

### Binding of Anti-LPS Antibody, 15308 in Adherent Cell Lines

The human lung epithelial cell line, A549 was grown on coverslips, fixed with 3% paraformaldehyde and permeabilised with 0.2% Triton X-100 in preparation for intracellular immunofluorescence. Cells were double-labeled with 15308 antibody and tubulin antibody (A). The distribution of 15308 antibody was visualised by secondary staining with alexaFluor-488 labeled secondary antibody (green). Microtubule distribution was detected by anti-β-tubulin antibody followed by alexaFluor-568 labeled secondary antibody (red). The microtubule and 15308 images were superimposed for assessment of colocalisation (gold appearance).Cells were double-labeled with 15308 antibody (green) and phalloidin labelled with alexaFluor-568(red) to show a distinct localisation from actin filaments (E). The results are shown in FIG. 7. The results show that the binding of anti-LPS antibody, 15308 localises specifically with microtubules in adherent cell lines.

### EXAMPLE 6

### Appearance of 15308 Epitope on the Surface of Human Lymphoma Cells During Apoptosis

The human B-cell lymphoma cell line, Mutu 1 was treated with ionomycin for 16h. Within the population, cells at various stages of death were discriminated on the basis of light scatter properties; Early (R1), middle (R2), late (R3). Staining with propidium iodide (PI) was used as a measure of membrane integrity. The appearance of 15308 epitope was analysed by staining with a FITC labeled goat anti-mouse secondary antibody. Staining with a gamma 3 isotype mouse monoclonal antibody (γ3) was included as a negative control. The results are shown in FIG. 8. The percentages of cells within each quadrant are shown. The percentages in the lower right corner represent 15308 positive and PI negative, apoptotic cells. As shown in the Figure, the 15308 epitope appears on the surface of human lymphoma cells during apoptosis.

### EXAMPLE 7

### The 15308 Epitope is Exposed on the Surface of Apoptotic Human Lymphoma Cells, as Determined by Confocal Microscopy

The human B-cell lymphoma cell line, Mutu 1 was treated with ionomycin for 16h to undergo apoptosis. Binding of the anti-LPS antibody, 15308 was detected with goat anti-mouse secondary antibody labeled with alexaFluor-488 (green). PI (red) was included in all samples as a measure the integrity of the membrane throughout the procedure and to exclude the possibility that the 15308 antibody bound intracellular epitopes instead of cell surface-exposed molecules. The results are shown in FIG. 9. As seen in A and B, the 15308 antibody stained the cell surface of some of the apoptotic cells (closed arrow heads), whereas secondary necrotic cells (open arrow heads) exhibited nuclear staining with PI and cytoplasmic staining. In C, different planes of surface fluorescent staining by 15308 are shown. The figure shows that the 15308 epitope is exposed on the surface of apoptotic human lymphoma cells, as determined by confocal microscopy.

### EXAMPLE 8

### Colocalisation of 15308 Reactivity and PS on the Surface of Apoptotic Human Breast Carcinoma Cells, as Determined by Confocal Microscopy

The cultured cell line, MCF-7 was treated with etoposide (100 μM) for 48 hours, and the colocalisation of the 15308 epitope and PS examined. Shown in FIG. 10 are the confocal fluorescence images of the 15308 epitope visualised by goat anti-mouse secondary antibody labelled with alexaFluor-568 (red), PS visualised by annexin V-biotin and streptavidin labelled with alexaFluor-488 (green).

The 15308 epitope and PS localise to regions containing bleb like structures (A and B)

A close up view showing staining of bleb surface (white square C) with single Z-section images (E,F and G)

Bright field (B and D)

Dual excitation for colocalisation (A,C and G)

As phosphatidyl serine(PS) is normally present only at the inner side of the cell membrane, these results demonstrate that alterations have taken place at the outer surface of the cell membrane. Without being limited to any one theory, it is believed that the results suggest that the appearance of the phosphatidyl serine at the outer surface of the membrane may be important in exposing the 15308 epitope on the outer surface of the membrane.

### EXAMPLE 9

### The 15308 Epitope Localises to the Surface Blebs of Apoptotic Human Breast Carcinoma Cells, as seen by Light and Fluorescence Microscopy

The cultured cell line, MCF-7 was treated with etoposide (100 μM) for 48 hours, and the surface distribution of the 15308 epitope was examined. Shown in FIG. 11 is a cell monolayer under bright field (A) or a fluorescence image of the 15308 epitope visualised by goat anti-mouse secondary antibody labeled with alexaFluor-488 (green).

### EXAMPLE 10

### Apoptotic Cells Bound with 15308 Antibody Induce Pro-Inflammatory Responses in Macrophages

Macrophages were treated with CD40L or anti-LPS mAb either alone or in the presence of apoptotic neutrophils. The results are shown in FIG. 12. Whereas CD40L stimulated a small increase in TNF production which was inhibited by apoptotic neutrophils (NP), opsonisation of the neutrophils with 15308 markedly induced TNF production by the macrophages. Thus, apoptotic cells bound with 15308 antibody induce pro-inflammatory responses in macrophages.

### EXAMPLE 11

### Binding of Anti-LPS Antibodies 15308 and 15174 to Primary Mouse Apoptotic Cells

A thymus was harvested from a mouse, dissociated by squashing between two frosted glass slides. The single cell suspension was induced to apoptosis with 25 μg/ml of dexamethasone (Sigma) and overnight incubation. Cells were then stained using indirect immunofluorescence techniques and analysed by flow cytometry techniques well known to people skilled in the art. The results of the staining with anti-LPS Ab 15308, anti-LPS Ab 15174 and secondary antibody alone are shown in FIG. 14 and provide further evidence that anti-microbial Abs can bind to apoptotic cells. This is the first evidence that anti-microbial Abs can bind to primary mouse cells. This binding would be, presumably, an essential pre-requisite for Abs that show therapeutic benefit.

### EXAMPLE 12

### In vivo Testing of Antibody

The animal models encompass both immunocompetent and immunocompromised animals which allow assessment of the relative importance of adaptive and innate anti-tumour immune responses.

As summarised in the schematic FIG. 13, the following tumours are subjected to treatment with monoclonal antibodies selected from the in vitro studies described above. The chosen tumours are those showing constitutive apoptosis together with macrophage infiltration.
1. Spontaneous λ-Myc tumours (histologically Burkitt-like with high-rate apoptosis and large numbers of starry-sky macrophages [Kovalchuk, 2000]);
2. Syngeneic transplantation of λ-Myc cell lines (lines already available);
3. Transplantation of λ-Myc cell lines to SCID or nude mice;
4. Transplantation of human BL lines to SCID or nude mice (these tumours closely resemble human BL histologically with frequent apoptotic cells and effective recruitment of host starry-sky macrophages).

As soon as tumours are detectable, the selected antibodies/antibody mixtures are administered via systemic or intratumoural routes (see below). Localisation of antibodies to apoptotic cells in tumours and in normal tissues are assessed histologically. Tumour transplantation approaches (2-4 above), will focus primarily on subcutaneous tumours which are not only easily monitored but are readily amenable to intratumour antibody administration.

Efficacy of antibodies in causing tumour regression is assessed by direct tumour measurement. Preliminary observations on underlying mechanisms are made in situ on histological material (for example, assessment of inflammatory infiltrate; macrophage activation) as well as on isolated immune cell populations in vitro. Anti-tumour immune responses are initially characterised using T-enriched lymphocytes from spleen or draining lymph nodes which will be tested for their ability to proliferate in vitro in response to tumour cells using standard methods. Cytotoxic T-cell activity within such responding T-cell populations are assessed against ⁵¹Cr-labelled tumour targets. Cytotoxic activity of TAMs from treated tumours are also be tested against tumour targets. In the event of absence of antibody-induced tumour regression, the aforementioned mechanistic studies will be invaluable in monitoring cause (eg lack of pro-inflammatory activation of TAMs).

### EXAMPLE 13

### Effect of Intra-Tumoural Injection of mAb 15174 or PBS on Tumour Growth

10⁷ BL2 cells were injected sub-cutaneously into Nude mice (supplied by Harlan) in a volume of 200 μl of PBS.

Appearance of tumours was monitored and measurements in two dimensions taken. As indicated by the arrows, tumours were directly injected with PBS (carrier) or mAb 15174 in PBS. 50 μl volume injected ip. mAb 15174 is an anti-LPS Ab available from QED Bioscience. Tumour size was followed for subsequent days until animals were sacrificed in line with Home Office regulations. A single injection of mAb 15174 shows a marked effect on tumour growth not seen with PBS injection. In animal #e21, tumour growth was halted after a single injection. NB—the injection on mouse #e17 was poor with some leak-back of Ab. This has subsequently not shown the degree of response seen with animal #e21 that received a full injection. Nevertheless, as with #e21, #e17 shows significant reduction in the rate of tumour growth after a single injection. Multiple injections should result in prolongation of the reduction/halting of the tumour growth and may result in regression.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

Aderem, A. A., Wright, S. D., Silverstein, S. C., and Cohn, Z. A. (1985). Ligated complement receptors do not activate the arachidonic acid cascade in resident peritoneal macrophages. J Exp Med 161, 617-622.

Arur, S., Uche, U. E., Rezaul, K., Fong, M., Scranton, V., Cowan, A. E., Mohler, W., and Han, D. K. (2003). Annexin I is an endogenous ligand that mediates apoptotic cell engulfment. Dev Cell 4, 587-598.

Berard, C., O'Conor, G. T., Thomas, L. B., and Torloni, H. (1969). Histopathological definition of Burkitt's tumour. Bulletin of the World Health Organisation 40, 601-607.

Carbone, A., Gloghini, A., Colombatti, A., Castronovo, V., and Menard, S. (1995). Expression of the monomeric 67-kd laminin-binding protein in human lymphomas as defined by MLuC5 monoclonal antibody and paraffin section immunohistochemistry. Hum Pathol 26, 541-546.

Caron, E., and Hall, A. (1998). Identification of two distinct mechanisms of phagocytosis controlled by different Rho GTPases. Science 282, 1717-1721.

Devitt, A., Moffatt, O. D., Raykundalia, C., Capra, J. D., Simmons, D. L., and Gregory, C. D. (1998). Human CD14 mediates recognition and phagocytosis of apoptotic cells. Nature 392, 505-509.

Devitt, A., Parker, K., Ogden, C. A., Oldreive, C., Clay, M. C., Hay, L., Bellamy, C. O., Lacey-Hulbert, A., Gangloff, S. C., Goyert, S., and Gregory, C. D. (2004). CD14 mediates tethering of apoptotic cells and can be functionally uncoupled from anti-inflammatory macrophage responses in apoptotic-cell clearance. Submitted.

Devitt, A., Pierce, S., Oldreive, C., Shingler, W. H., and Gregory, C. D. (2003). CD 14-dependent clearance of apoptotic cells by human macrophages: the role of phosphatidyiserine. Cell Death and Differentiation 10, 371-382.

Dive, C., Gregory, C. D., Phipps, D. J., Evans, D. L., Milner, A. E., and Wyllie, A. H. (1992). Analysis and discrimination of necrosis and apoptosis (programmed cell death) by multiparameter flow cytometry. Biochimica Et Biophysica Acta 1133, 275-285.

Dziarski, R., Ulmer, A. J., and Gupta, D. (2000). Interactions of CD14 with components of Gram-positive bacteria. In CD14 in the Inflammatory Response, pp. 83-107.

Fadok, V. A., de Cathelineau, A., Daleke, D. L., Henson, P. M., and Bratton, D. L. (2001). Loss of phospholipid asymmetry and surface exposure of phosphatidylserine is required for phagocytosis of apoptotic cells by macrophages and fibroblasts. Journal of Biological Chemistry 276, 1071-1077.

Flora, P. K., and Gregory, C. D. (1994). Recognition of apoptotic cells by human macrophages—inhibition by a monocyte/macrophage-specific monoclonal antibody. European Journal Of Immunology 24, 2625-2632.

Fujii, C., Shiratsuchi, A., Manaka, J., Yonehara, S., and Nakanishi, Y. (2001). Difference in the way of macrophage recognition of target cells depending on their apoptotic states. Cell Death and Differentiation 8, 1113-1122.

Gordon, S. (2003). Alternative activation of macrophages. Nature Reviews Immunology 3, 23-35.

Gregory, C. D. (2000). CD14-dependent clearance of apoptotic cells: relevance to the immune system. Current Opinion in Immunology 12, 27-34.

Gregory, C. D., and Devitt, A. (1999). CD14 and apoptosis. Apoptosis 4, 11-20.

Harris, N. L. (1995). A practical approach to the pathology of lymphoid neoplasms: a revised European-American classification from the International Lymphoma Study Group. In Important Advances in Ocology 1995, V. T. deVita, S. Hellman, and S. A. Rosenberg, eds. (Philadelphia, Lippincott Company), pp. 111-140.

Henson, P. M., Bratton, D. L., and Fadok, V. A. (2001). Apoptotic cell removal. Current Biology 11, R795-R805.

Hori, M., Xiang, S., Qi, C. F., Chattopadhyay, S. K., Fredrickson, T. N., Hartley, J. W., Kovalchuk, A. L., Bornkamm, G. W., Janz, S., Copeland, N. G., et al. (2001). Non-Hodgkin lymphomas of mice. Blood Cells Molecules and Diseases 27, 217-222.

Kazmin, D. A., Chinenov, Y., Larson, E., and Starkey, J. R. (2003). Comparative modeling of the N-terminal domain of the 67kDa laminin-binding protein: implications for putative ribosomal function. Biochem Biophys Res Commun 300, 161-166.

Kitchens, R. L. (2000). Role of CD14 in cellular recognition of bacterial lipopolysaccharides. In CD14 in the Inflammatory Response, pp. 61-82.

Kovalchuk, A. L., Qi, C. F., Torrey, T. A., Taddesse-Heath, L., Feigenbaum, L., Park, S. S., Gerbitz, A., Klobeck, G., Hoertnagel, K., Polack, A., et al. (2000). Burkitt lymphoma in the mouse. Journal of Experimental Medicine 192, 1183-1190.

Mantovani, A., Sozzani, S., Locati, M., Allavena, P., and Sica, A. (2002). Macrophage polarization: tumor-associated macrophages as a paradigm for polarized M2 mononuclear phagocytes. Trends in Immunology 23, 549-555.

Moffatt, O. D., Devitt, A., Bell, E. D., Simmons, D. L., and Gregory, C. D. (1999). Macrophage recognition of ICAM-3 on apoptotic leukocytes. Journal Of Immunology 162, 6800-6810.

Ronchetti, A., Rovere, P., Iezzi, G., Galati, G., Heltai, S., Protti, M. P., Garancini, M. P., Manfredi, A. A., Rugarli, C., and Bellone, M. (1999). Immunogenicity of apoptotic cells in vivo: Role of antigen load, antigen-presenting cells, and cytokines. Journal Of Immunology 163, 130-136.

Sauter, B., Albert, M. L., Francisco, L., Larsson, M., Somersan, S., and Bhardwaj, N. (2000). Consequences of cell death: Exposure to necrotic tumor cells, but not primary tissue cells or apoptotic cells, induces the maturation of immunostimulatory dendritic cells. Journal of Experimental Medicine 191, 423-433.

Savill, J., Dransfield, I., Gregory, C. D., and Haslett, C. (2002). A blast from the past: clearance of apoptotic cells regulates immune responses. Nature Reviews Immunology 2, 965-975.

Stein, M., and Gordon, S. (1991). Regulation of tumor necrosis factor (TNF) release by murine peritoneal macrophages: role of cell stimulation and specific phagocytic plasma membrane receptors. Eur J Immunol 21, 431-437.

van Meyel, D. J., Sanchez-Sweatman, O. H., Kerkvliet, N., Stitt, L., Ramsay, D. A., Khokha, R., Chambers, A. F., and Cairncross, J. G. (1998). Genetic background influences timing, morphology and dissemination of lymphomas in p53-deficient mice. International Journal of Oncology 13, 917-922.

Watanabe, Y., Shiratsuchi, A., Shimizu, K., Takizawa, T., and Nakanishi, Y. (2002). Role of phosphatidylserine exposure and sugar chain desialylation at the surface of influenza virus-infected cells in efficient phagocytosis by macrophages. Journal of Biological Chemistry 277, 18222-18228.

Wright, S. D., and Silverstein, S. C. (1983). Receptors for C3b and C3bi promote phagocytosis but not the release of toxic oxygen from human phagocytes. J Exp Med 158, 2016-2023.

Zenger, E., Abbey, N. W., Weinstein, M. D., Kapp, L., Reis, J., Gofman, I., Millward, C., Gascon, R., Elbaggari, A., Herndier, B. G., and McGrath, M. S. (2002). Injection of human primary effusion lymphoma cells or associated macrophages into severe combined immunodeficient mice causes murine lymphomas. Cancer Research 62, 5536-5542.

1. A method of enhancing an immune response to apoptotic cells, said method comprising administration of a specific binding member which specifically binds to a microbial antigen.

2. A method of treating cancer comprising administration of a therapeutically effective amount of a specific binding member or a nucleic acid encoding said specific binding member which specifically binds to a microbial antigen to a subject in need thereof.

3. An assay method for identification of an antibody capable of enhancing an immune response to apoptotic cells, said assay method comprising the steps:
(a) bringing into contact a specific binding member with binding specificity for a microbial antigen with an apoptotic cell; and
(b) determining the absence or presence of cross-reactivity of the specific binding member with the apoptotic cell, wherein the presence of cross-reactivity of the specific binding member with the apoptotic cell is indicative of ability to enhance an immune response against said apoptotic cells.

4. The assay method according to claim 3, wherein the method further comprises step(c) comprising bringing together (i) apoptotic cells and the specific binding member determined in step (b) to show crossreactivity with the apoptotic cells and (ii) innate immune cells and the induction of an immunostimulatory response, and/or inhibition of an immunosuppressive response, in the innate immune cells.

5. The method according to claim 4, wherein the innate immune cells are macrophages.

6. An assay method for identification of a specific binding member capable of enhancing an immune response to apoptotic cells, said assay method comprising steps:
(a) providing a specific binding member with binding specificity for a microbial antigen
(b) bringing into contact said specific binding member, apoptotic cells and macrophages, and
(c) determining the induction of an immunostimulatory response, and/or inhibition of an immunosuppressive response, in the macrophages, wherein the presence of an immunostimulatory response and/or inhibition of an immunosuppressive response is indicative of ability to enhance an immune response against said apoptotic cells.

7. The assay method according to claim 4, claim 5 or claim 6, wherein the method further comprises step (d) identifying the apoptotic cell epitope to which the antibody binds.

8. The method according to claim 1, 2, 3 or 6, wherein the specific binding member has cross-reactivity with an apoptotic cell epitope.

9. The method according to claim 8, wherein the specific binding member has binding specificity for a ligand which binds CD14.

10. The method according to claim 9, wherein the ligand which binds CD14 is an LPS, lipotecheic acid, peptidoglycan or lipoarabinomannan ligand.

11. The method according to claim 1, 2, 3 or 6, wherein the specific binding member is mAb wn1 222-5, mAb 15174, mAb 15306, mAb 15308, mAb 983, mAb 995, mAb 756, or mAb 746 MAB 8598.

12. (canceled)

13. (canceled)

14. A pharmaceutical composition for the treatment of cancer, wherein the composition comprises a specific binding member which specifically binds to a microbial antigen or a nucleic acid encoding said specific binding member.

15. (canceled)

16. (canceled)

17. (canceled)

18. (canceled)

19. A method of enhancing an immune response to apoptotic cells, said method comprising administration of a specific binding member which specifically binds to an intracellular antigen.

20. A method of treating cancer, said method comprising administration of a therapeutically effective amount of a specific binding member which specifically binds to an apoptotic cell intracellular antigen to a subject in need thereof.

21. (canceled)

22. (canceled)

23. A pharmaceutical composition for the treatment of cancer, wherein the composition comprises a specific binding member which specifically binds to an apoptotic cell intracellular antigen.

24. The method according to claim 19 or 20, wherein the specific binding members having binding specificity for actin or phosphatidyl serine.

25. A method of diagnosing the presence of apoptotic cells in a biological sample, said method comprising the steps:
(a) bringing into contact a specific binding member which specifically binds to a microbial antigen and a biological sample;
(b) determining the presence of binding of the specific binding member to cells of the biological sample; wherein the presence of binding of the specific binding member to cells of the biological sample is indicative that the cells being bound are undergoing apoptosis.

26. A method for monitoring the progression of a cancer in a patient, comprising the steps of:
(a) contacting a biological sample from a patient at a first point in time with specific binding member which specifically binds to a microbial antigen;
(b) detecting in the sample an amount of specific binding member that binds to the binding agent;
(c) repeating steps (a) and (b) using a biological sample obtained from the patient at a subsequent point in time; and
(d) comparing the amount of binding member detected in step (c) to the amount detected in step (b) and therefrom monitoring the progression of the cancer in the patient.

27. A method of detecting the presence of apoptotic cells in tissue in vivo, said method comprising the steps
(a) bringing into contact a specific binding member which specifically binds to a microbial antigen and a said tissue in vivo;
(b) determining the presence of binding of the specific binding member to cells of the tissue; wherein the presence of binding of the specific binding member to cells of the tissue is indicative that the cells being bound are apoptotic.

28. A method for monitoring the progression of a cancer in a patient, comprising the steps of:
(a) contacting a tissue in vivo at a first point in time with a specific binding member which specifically binds to a microbial antigen;
(b) detecting in the amount of specific binding member that binds to the tissue;
(c) repeating steps (a) and (b) at a subsequent point in time; and
(d) comparing the amount of binding member detected in step (c) to the amount detected in step (b) and therefrom monitoring the progression of the cancer in the patient.

29. The method according to any one of claims 25 to 28, wherein the specific binding member is mAb wn1 222-5, mAb 15174, mAb 15306, mAb 15308, mAb 983, mAb 995, mAb 756, or mAb 746 MAB 8598.

30. (canceled)

31. A diagnostic kit for the diagnosis of cancer, said kit comprising (i) a specific binding member which specifically binds to an apoptotic cell intracellular antigen or (ii) a nucleic acid encoding said specific binding member.
